(19) United States
(12) Patent Application Publication
(10) Pub. No.: US 2005/0208101 A1
(43) Pub. Date: Sep. 22, 2005

(54) Coating composition for an implantable medical device and method for coating such a device

(76) Inventor: Viktor Sevastianov, Moskau (RU)

Correspondence Address: MORRISON & FOERSTER LLP, 3811 VALLEY CENTRE DRIVE, SUITE 500, SAN DIEGO, CA, 92130-2332, US

(21) Appl. No.: 11/041,042

(22) Filed: Jan. 21, 2005

Related U.S. Application Data
(63) Continuation of application No. PCT/EP03/07913, filed on Jul. 19, 2003.

(30) Foreign Application Priority Data
Jul. 23, 2002 (DE) 102 34 398.5

(51) Int. Cl. A61F 2/06; A61F 2/02; B05D 1/02; B05D 1/18

(52) U.S. Cl. 424/426; 623/1.46; 427/2.25; 427/2.21

(57) ABSTRACT

The invention relates to a coating composition for an implantable medical device. The coating composition, which in this connection comprises at least one polymer and at least one bioactive agent, includes as bioactive substance naphthoquinone and/or a naphthoquinone derivative, in particular shikonin.

The present invention relates to a coating composition for an implantable medical device, where the coating includes at least one polymer and at least one bioactive agent.

Various coating compositions and methods for coating implantable medical devices are sufficiently well known in the art.

Coated implantable medical devices are used for example as skin, bone or cartilage substitute and are very important as prostheses especially in vascular surgery.

Prostheses of these types are implanted in a lumen of the body, for example in a blood vessel, of a patient in order to replace these vessels for the relevant fluids over a defined distance—for example in the case of a vessel prosthesis—or in order to widen them and keep them open by a so-called stent. In such cases, the prostheses, which usually have a cylindrical shape, support the lining of the vessel and prevent the vessels from collapsing or their lining blocking the passage through the vessels. Materials conventionally used for prostheses are, for example, synthetic materials such as woven filaments of polyethylene terephthalate (PET) or of expanded polytetrafluoroethylene (ePTFE), but various metals are also employed in addition.

It is not necessary in every case of a vessel constriction or a vascular occlusion (stenosis) to perform an invasive surgical procedure. Even in cases of occlusion of the coronary vessels of the heart it is possible in many cases to avoid a heart operation with opened chest through a prosthesis which is introduced through vessels (intravascularly). For this purpose, for example, a catheter which has on its tip an inflatable balloon and an expandable mechanical support for the vessel (stent) is introduced via a vein as far as into the vessel constriction. The vessel and stent are simultaneously expanded through the inflation of the balloon; the flow of blood through the stenosis thus becomes possible again.

This rather forcible widening is associated with injury to the vessel. It is supported against collapse by the rigid stent. Even if the stent has a very open structure in the deployed state, there are many points of contact between the rigid supporting materials and the injured vessel wall. This gives rise to two consecutive problems:

All vessel injuries have thrombogenic effects. Via activation of the blood platelets by vessel material under the endothelium there is formation in the lumen of a thrombus which is intended to prevent escape of blood. Foreign surfaces such as metals or plastics from which stents are produced also have thrombogenic effects and may through the thrombus formation lead within a short time to renewed vascular occlusion (restenosis).

During healing of the vessel which has been damaged by the widening, there is moreover scar formation especially where a permanent stress is exerted on the regenerating tissue by points of pressure from the rigid stent. Excessive proliferation of scar tissue leads to restenosis with about 30% of all uncoated stents after a relatively long time.

Coating of the stent, which is necessarily rigid as supporting material, is intended to prevent both risks of restenosis—the short-term risk of thrombi and the long-term proliferation of scar tissue.

These problems, namely the avoidance of coagulation and proliferation and the prevention of activation of blood platelets, are approached in different ways in the prior art. Such research aims for example are coatings of stents which are intended to provide increased hemocompatibility. For example, anticoagulant, antimicrobial, antiinflammatory or antiproliferative agents, which are generally referred to as “bioactive” substances, have been employed for a relatively long time in the coating of stents. These substances are intended to be released from the coating material of the stent in such a way that they prevent inflammations of the surrounding tissue, excessive growth of smooth muscle cells or dotting of blood.

U.S. Pat. No. 5,788,979 discloses a method for coating a biocompatible material which comes into contact with a patient's blood, the composition of the coating being intended to prevent coagulation of the blood by the biomaterial. In the method, initially a biodegradable material which is compatible with blood and tissue of the human body is prepared in a liquid state, and subsequently an anticoagulant composition is put into the liquid, biodegradable material. A liquid coating material which can be applied in a continuous manner to a biocompatible material, and subsequently dried, is produced in this way. Layer thicknesses of less than 100 μm can be produced with this method and the materials used.

U.S. Pat. No. 5,788,979 further discloses that the biodegradable material can be in particular a biodegradable, synthetic polymer such as, for example, polyglycolic acids, polylactic acids, polyhydroxybutyrates, polyhydroxyvalerates, polydioxanones, modified starches, celluloses etc.

It is additionally proposed that, besides the anticoagulant composition, further substances may be present in the coating, such as, for example, antiinflammatory, antiproliferative, antibiotic substances. Examples thereof mentioned in the patent are dexamethasone, gentamycin and hirudin.

A disadvantage of said coatings is that on use of anticoagulant compositions the respective administration and dosage must be observed most carefully and be suited to the individual patient, because these substances are always associated with a high risk of acute hemorrhages. In addition, after contact with recombinantly produced hirudin, many patients develop antibodies against hirudin-thrombin complexes, making the antithrombotic effect of this substance uncontrollable. The use of glucocorticoids such as dexamethasone causes, especially over relatively long periods, the side effects on protein and carbohydrate metabolism which are normally observed with these hormones.

DE 195 21 642 discloses implants which consist at least partly of an absorbable material and which have in this absorbable material an antibiotic active substance, this active substance being released into the surroundings throughout the breakdown phase of the absorbable material. The antibiotic active substance mentioned in this patent is in particular gentamycin.

A disadvantage of the use of gentamycin is that there are high individual variations in its therapeutic and toxic concentration. Thus, side effects such as nephrotoxicity, neuronal blocks and, in particular, vestibular and cochlear impairments have been described in connection with gentamycin.

Further compounds which may be mentioned in this connection are the cytostatics Taxol (paclitaxel) and rapamycin and derivatives thereof. However, many side effects and complications on use as stent coating are also known from the literature for these, e.g. the high thrombogenicity of Taxol (F. Liistro, A. Colombo, “Late acute thrombosis after paclitaxel eluting stent implantation”, Heart, 86: 262-264 (2001)).

On use of rapamycin as stent coating (U.S. 2001 027 340), current clinical studies have shown only a limited reduction of restenosis (Cordis SIRIUS study: 10% restenosis). Besides the high toxicity of these cytostatics, disadvantages are the chemical instability and the resulting difficulty of accurate dosage. Thus, some patients in the abovementioned study have also shown symptoms of overdosage (thinning of the vessel wall).

A further risk for example with the coated stents known in the art is that the coating material is damaged during processes of adaptation of the stent to the relevant vessel—in the form of expansions or compressions. The coating and the release of the bioactive substances are thereby impaired in their respective function and effect.

There is further a great need to be able to control more simply and more accurately the delivery of the bioactive substance in a coating to the surroundings of the implant.

Despite the various degradable coating materials frequently suggested in the prior art, and despite the large number of compositions having antibiotic, antimicrobial or anticoagulant activity which have been investigated in this connection, restenoses of vessels and rejection reactions of implants remain a great problem. Accordingly, despite all this there is a great need for devices which can keep, for example, vessels permanently free, and which provide for good bioresorption.

It is therefore an object of the present invention to provide a coating which includes a polymer with a novel biocompatible bioactive agent, while preferably—with an optimization of the mechanical properties of the coating—defined amounts of this bioactive substance can be incorporated and its release from the coating can be controlled in a simple manner.

According to the present invention, this object is achieved with the coating composition mentioned at the outset in that the bioactive agent is naphthoquinone and/or a naphthoquinone derivative.

The object on which the invention is based is completely achieved in this way.

The inventor has realized that it is possible through the use of polymers and of naphthoquinone in a coating to produce an implantable medical device which has excellent mechanical properties, i.e. can withstand both compressions and expansions, and that the incorporation of naphthoquinones has no adverse effect on the mechanical properties, rather the substance is retained as bioactive agent.

The inventor has further realized that it is possible through the use of certain naphthoquinones to check visually whether, and to what extent, the coating of implantable medical devices has suceeded. This is achieved through the coloring inherent in the naphthazarin derivatives: if a coating comprises a naphthoquinones, it is possible to ascertain on the basis of the coloring whether a device is uncoated, coated or only partly coated.

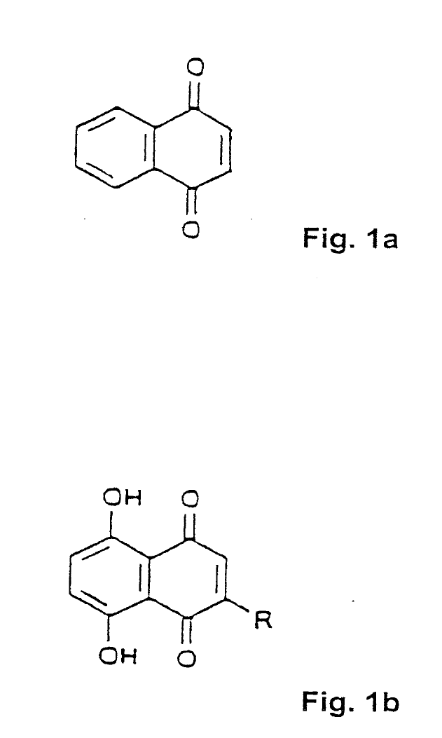

Naphthoquinones are oxidation products of naphthalene comprising a quinoid constitution of the carbonyl groups. By way of example, in FIG. 1a the general formula of substance 1,4-naphthoquinone is shown as a representative of naphthoquinones.

In a preferred embodiment, the naphthoquinone derivative is naphthazarin and/or a naphthazarin derivative.

Naphthazarin is known in the art as a basic structure in many natural pigments which additionally also represent medicinal agents. It is possible through the use of naphthazarin and/or naphthazarin derivatives to achieve two advantageous effects with one active substance. Firstly, these natural products are colored, so that the success of the coating can be checked visually, and secondly they simultaneously have a healing effect, which makes the addition of further bioactive substances in the coating unnecessary.

In this connection, “naphthazarin derivatives” comprise all substances, which comprise naphthazarin as basic structure, the general formula of which is shown in FIG. 1b.

Naphthazarin derivatives in the present case include all compounds which have the basic structure of naphthazarin, while the radical R for example can be any aliphatic radical which may be acyclic or cyclic, unbranched or branched, or in substituted (for example hydroxy-substituted) form.

In a further embodiment, the derivative is selected from the group comprising shikonin, alkannin, arnebin and derivatives thereof. Particularly preferred in this connection is shikonin. The inventor was able to show in his own experiments that prevention of blood platelet and fibroblast aggregation was possible with stents coated with the naphthazarin derivative shikonin.

The range of effects of shikonin is considerably broader by comparison with the compounds previously used in connection with coatings, such as, for example, rapamycin and Taxol. Shikonin, alkannin and derivatives thereof have been known for some time as red natural pigments and as medicinal agents. Thus, for example, Papageorgiou et al., “Chemie und Biologie von Alkannin, Shikonin und verwandten Naphthazarin-Naturstoffen”, Angew. Chemie 111: 280-311, 1999, present in their review article the biological and pharmacological properties which have been known for a relatively long time for these agents, and discuss bioorganic, preparative and medical aspects: thus, the article cites other publications in which it was shown that shikonin itself has an antiinflammatory and antimicrobial effect. The article by Papageorgiou et al. additionally cites a publication in which an antithrombotic effect of a few naphthazarin derivatives is described, but this effect could not be unambiguously ascribed to the shikonin family.

Shikonin has been proved to have an antitumor effect, and is antimycotic, antimicrobial and wound-healing. The cytostatics (for example rapamycin or Taxol) used to date do not achieve the unique profile of effects of shikonin.

Hisa et al., “Shikonin, an Ingredient of Lithospermum Erythrorhizon, Inhibits Angiogenesis in Vivo and in Vitro”, Anticancer Res. 18: 783-790, 1998, showed that shikonin was able to inhibit the proliferation of bovine endothelial cells, leading to inhibition of angiogenesis. By contrast, this article does not describe or explain whether, and how, shikonin or substances related to shikonin are able to prevent the proliferation of fibroblasts, especially of myofibroblasts, whose proliferation is, as previously mentioned, involved in the restenosis of vessels.

The results shown by the inventor were not to be expected especially taking account of the article by Sakaguchi et al., “Granulomatous Tissue Formation of Shikon and Shikonin by Air Pouch Method”, Biol. Pharm. Bull 24(6): 650-655, 2001, in which it is explained that Shikonin stimulates neoangiogenesis. On the contrary, the known properties of shikonin have to date been a deterrent to the use of shikonin or other naphthoquinone or naphthazarin derivatives in connection with implantable medical devices. The inventor was the first to be able to show that shikonin is able to prevent the aggregation of blood platelets and thus both thromboses and long-term restenoses.

Naphthoquinones have not previously been proposed for coating implantable medical devices.

In addition, naphthoquinones or naphthazarin derivatives are suitable for better examination of the kinetics of release in development. Other colorants, for example, without any biological activity, would merely be further foreign substances in the coating and would thus increase the risk of an allergic or inflammatory reaction. Accordingly, the use of shikonin or of other naphthoquinone derivatives or naphthazarin derivatives provides a final visual check of whether, and how well, an implant has been coated.

It is not precluded in this connection that the coating composition also includes a plurality of naphthoquinone derivatives or naphthazarin derivatives, or else further concomitant substances are also incorporated into the coating composition, such as, for example, anticoagulant, antimicrobial or antiinflammatory substances.

In a further preferred embodiment, the coating composition comprises naphthoquinone and/or a naphthoquinone derivative in a content of from 0.01 to 1% by weight, preferably of 0.5% by weight.

The inventor has been able to show in his own experiments that the use of from 0.1 to 1% by weight of a naphthoquinone, especially shikonin, in the coating is suitable for exerting an adequate inhibitory effect on blood platelets and fibroblast adhesion.

In a further embodiment, it is preferred for the polymer to be an absorbable polyester and to be selected in particular from the group comprising polyglycolic acid, polylactic acid, polycaprolactone, polyhydroxyalkanoates and copolyesters thereof.

Such absorbable polyesters and copolyesters are sufficiently well known in the art and have proved to be sufficiently useful in particular in medical uses.

It is particularly preferred in this connection for the polyhydroxyalkanoate to be selected from the group comprising polyhydroxybutyrate, polyhydroxyvalerate and copolyesters thereof.

The inventor has been able to show in his own experiments that, in particular, copolyesters of polyhydroxybutyrate and polyhydroxyvalerate have outstanding properties as coating material. These polyesters ensure that the coating is both biodegradable and has excellent mechanical properties. It further emerged from his own experiments that a naphthazarin derivative incorporated into this copolyester showed excellent biological activities.

It is known that many organic biodegradable polymers are able to release active substances within a certain time window. However, many materials are unsuitable as coating because they are not entirely biocompatible. Thus, for example, van der Giessen et al., “Marked Inflammatory Sequelae to Implantation of Biodegradable and Nonbiodegradable Polymers in Porcine Coronary Arteries”, Circulation 94: 1690-1697, 1996, showed that polylactides and other polymers thought to be biocompatible led to inflammatory tissue reactions on degradation in the body.

It is additionally known that many biodegradable polymers do not have appropriate mechanical properties. Thus, for example, crystalline regions may lead to sudden fissuring. For these reasons, a coating for an implantable medical device must have particular mechanical properties, and withstand both compression and expansion—for example an inflation of the catheter. In a further embodiment, the copolyester comprises polyhydroxyvalerate in a content of from 20 to 30% by weight, preferably of 25% by weight, and polyhydroxybutyrate in a content of from 70 to 80% by weight, preferably of 75% by weight.

The inventor was able to show in his own experiments that this ratio is particularly suitable for use as coating material, because good solubility is ensured with this ratio and, at the same time, the excellent mechanical properties of the polyester are attained.

It is further preferred in a further embodiment for the absorbable polymer and the shikonin to be preferably dissolved in at least one solvent, preferably dimethylacetamide and/or tetrahydrofuran.

It could be shown that the mechanical and biological properties of the coating are not impaired by dissolving the active components in these solvents.

The invention further relates to a method for coating an implantable medical device comprising the steps:
(a) applying the novel coating composition to the implantable medical device,
(b) drying of the implantable medical device and
(c) where appropriate repetition of steps (a) and (b).

In one embodiment it is preferred in this connection for the coating composition to be sprayed onto the implantable medical device.

Various techniques can be used for the spraying, all of which are sufficiently well known in the art.

In another preferred embodiment, the coating is applied by immersing the implantable medical device into the coating.

The coating processes are moreover repeated until the desired layer thickness for the coating on the implantable medical device is reached, for example a layer thickness of from 1 to 100 μm.

A preferred embodiment of the method of the invention for coating an implantable medical device consists of applying a coating which includes a polyhydroxybutyrate-polyhydroxyvalerate copolyester in which the polyhydroxybutyrate:polyhydroxylvalerate ratio is 3:1 and in which shikonin is present in a content of from 0.01 to 1% by weight, preferably of 0.5% by weight.

The inventor has realized that it is possible with this composition to produce a particularly suitable coating with which, besides the optimal mechanical properties of a coating, it is also possible to utilize effectively the dual function of shikonin—as colorant and bioactive agent. The properties of shikonin—colorant and active agent—are retained even after incorporation into the coating according to the invention.

In a further preferred embodiment, a stent is employed as device in the method for coating an implantable medical device.

It is possible in this connection to employ for example stents which include at least one metal and/or one synthetic material. Stents of these types, and methods for coating stents of these types, are sufficiently well known in the art.

However, it is not precluded that other types of implantable medical devices can be coated with the coating composition of the invention. Thus, for example, skin implants, a cartilage or bone substitute are also suitable, which may be flat, rectangular, cylindrical or configured as valve.

If a vessel prosthesis is used as implantable medical device, the tubular design thereof can be of any shape, that is to say for example as branched or unbranched tube etc.

The invention further relates to the use of a novel coating composition as indicated above for coating implantable medical devices.

The invention further relates to the use of naphthoquinone and/or naphthoquinone derivatives, especially of shikonin, for producing a coating composition for an implantable medical device and to the use for coating an implantable medical device.

The invention further relates to an implantable medical device which is coated with a novel coating composition as mentioned above, and especially stents.

Further advantages are evident from the description hereinafter.

It will be understood that the features mentioned above and to be explained below can be used not only in the combinations indicated in each case, but also in other combinations or on their own, without leaving the scope of the present invention.

The invention is explained in more detail below by means of use and implementation examples and by the figures. In these

FIG. 1a shows the general formula of the substance 1,4-naphthoquinone; and

FIG. 1b shows the general formula of the basic structure of naphthazarin.

### EXAMPLE

1. Coating Compositions Used

The following polymer compositions were tested as coating material:
a composite coating of the non-absorbable polyurethane Elastollan, with and without shikonin;
a biodegradable copolyester of poly(β-hydroxybutyrate-co-β-hydroxyvalerate) (P(HB-HV) hereinafter) with a content of 12% by weight polyhydroxyvalerate;
a biodegradable copolyester of P(HB-HV) with a content of 25% by weight polyhydroxyvalerate, with and without shikonin.

The solvents tested with dimethylacetamide (DMAA hereinafter) and tetrahydrofuran (THF hereinafter).

It emerged from this that a composition of 25% by weight polyhydroxyvalerate (PHV hereinafter) in relation to 75% polyhydroxybutyrate (PHB hereinafter) is most suitable. In addition, a composition with this ratio in both solvents with a maximum concentration of up to 1.5% by weight could be dissolved in both solvents by heating.

In this connection, a working solution (I) comprising P(HB-HV) with 25% by weight PHV with the following composition was used for the coating composition:
P(HB-HV): 1.5 g
DMAA or THF: 100 ml

Besides this pure copolyester, coating with another composition, namely P(HB-HV) with a content of 25% by weight PHV, and with shikonin, was tested. The working solution (II) used for this had the following composition:
P(HB-HV): 1.5 g
shikonin: from 1.5 to 7.5 mg
DMMA or THF: 100 ml.

The tests were carried out using firstly stents (made of stainless steel with electropolished surface, Translumina GmbH, Hechingen, Germany) coated with working solutions I and II, and secondly polymer films from indicated working solutions I and II cast in Petri dishes.

2. Cell Adhesion and Cell Proliferation Studies

a) Elastollan Films

Materials and Methods

Elastollan films containing 0.1 and 0.5% by weight shikonin, and Elastollan films without shikonin, were tested.

Although Elastollan is a non-absorbable polymer, nevertheless the results obtained with this polymer show the activity of shikonin as inhibitor of cell adhesion and proliferation. This means that in this case the active agent is released by leaching and not by degradation of the Elastollan matrix.

Human embryonic fibroblasts were employed in these experiments. The cells were cultivated in Eagle's medium with the addition of 10% fetal calf serum and passaged twice a week using a trypsin-EDTA solution. The cells present after the 14th passage were used for the tests.

Mitomycin C dissolved in culture medium in a concentration of 20 μg/ml was used as negative control.

Extracts from Elastollan films without shikonin, Elastollan films with 0.1% by weight and 0.5% by weight shikonin (based on polyurethane), and shikonin alone, were tested.

Extracts were obtained by incubating dishes covered by the polymers to be tested (i.e. Elastollan films without or with shikonin) with Eagle's medium at 37° C. for 3 h. Undissolved solids were removed by filtering. The control with shikonin only (in the culture medium) was cultivated under the same conditions.

The respective filtrates were employed as test extracts, with the pH of the shikonin containing Elastollan extracts having been adjusted previously with 0.1 N HCl, and with the same amount of phosphate buffer as was necessary for adjusting the pH of the Elastollan/shikonin extracts having been added to the control extract (Elastollan without shikonin) and to the extract with shikonin alone.

In parallel, 1 ml portions of the cell suspension (fibroblasts, see above) with 4×10⁴ cells/ml were applied to 24-well plates. After a cultivation time of 24 hours, the medium was removed and the extracts to be tested were added (positive control: fresh medium without extracts; negative controls: addition of mitomycin C for 2 h).

The cells were then washed with Hank's solution, and fresh medium was put into the wells. The cells were then incubated for 72 h. After this incubation time, the cultures were washed twice with phosphate buffer and incubated with 2.5% glutaraldehyde at 4° C. for 30 min. They were then washed again twice and stained with Giemsa at 37° C. in humid atmosphere for 3 h.

The stain retained by the cells was eluted with a phosphate buffer/alcohol mixture (1:1) at room temperature for 15 min. The optical density of the resulting solutions was determined by a spectrophotometer with a wavelength of 620 nm.

Results

In the positive control (tested extract: only culture medium), the fibroblasts covered almost the entire surface of the well and showed an elongate shape and a typical growth pattern.

In the negative control (tested extract: culture medium+mitomycin C), no cell growth was observed. The cell density was low and corresponded to that on the second day of the experiment. The cells had an elongate shape but were not in contact with one another. Some cells were rounded or were lyzed.

Cell growth on extract from Elastollan alone was similar to that in the positive control. On use of shikonin-containing Elastollan extracts it was possible to observe an inhibitory and even cytotoxic effect: a few cells were adherent, most were rounded.

This test showed that shikonin and extracts from shikonin-containing Elastollan films have an inhibitory effect on human cells in vitro.

B) In Vitro Adhesion of Human Embryonic Lung Fibroblasts to Elastollan- and Shikonin-Containing Elastollan Films

Petri dishes coated with films of Elastollan- and shikonin-containing Elastollan-DMAA solutions were used for this test. The concentration of shikonin in the polyurethane samples was 0.01% by weight, 0.05% by weight and 0.5% by weight (based on polyurethane). A plastic Petri dish served as positive control.

The cells were seeded on the surface of the Petri dishes coated with the polymer compositions in a concentration of 4×10⁴ cells/ml in Eagle's medium with 10% fetal calf serum. The number of adherent fibroblasts was counted after incubation at 37° C. for 0.5 and 2 h.

Results

The counts obtained are shown in table I below.

As is evident from the table through comparison with the positive control, a concentration of 0.5% by weight shikonin in the Elastollan films inhibits the adhesion of fibroblasts virtually completely:

Number of counted fibroblasts
(after an incubation time of)

| Type of surface | 0.5 h | 2 h |
|---|---|---|
| Control | ca. 60 | ca. 95 |
| (plastic Petri dish) | | |
| Elastollan | 10-40 | none |
| Elastollan + shikonin | <20 | none |
| (0.01% by weight) | | |
| Elastollan + shikonin | ca. 30 | none |
| (0.05% by weight) | | |
| Elastollan + shikonin | 0-5 | none |
| (0.5% by weight) | | |

C) Adhesion of Blood Platelets onto Copolyester P(HB-HV) Films and onto Shikonin-Containing Copolyester P(HB-HV) Films (P(HB-HV)-Sh) and onto Stents Coated with These Films

It is known that the adhesion of blood platelets to biomaterials reflects the compatibility of medical devices with blood in relation to blood and tissue cell activation.

It is presumed that adhesion proceeds in a plurality of steps: initially the blood platelets bind to the surface, are then activated and develop pseudopods, and then they spread out and form aggregates. The subsequent release of intracellular components, including blood clotting factors, stimulates adhesion and aggregation of further blood platelets.

Besides, infiltration of actively proliferating myocytes from the media into the intima, accompanied by the production of abundant extracellular matrix components (collagen, proteoaminoglycans), is presumed to be the fundamental mechanism of restenosis. Immediate deposition of blood platelets at the point where the vessel was injured, and the subsequent release of myoproliferative substances (for example the growth factor (PDGF), β-transforming factor (β-TGF), endothelium-derived growth factor (EDGF), etc.) probably represent the stimulating factors.

Thus, adhesion of blood platelets during stent implantation plays a key role both in relation to thromboses and to restenoses.

The activation status of adherent blood platelets can be estimated from their morphology. A larger effect of the material on blood platelets results in more adherent cells being distributed or aggregated on the material.

Both uncoated stents and stents coated with P(HB-HV)-Sh were tested for this test. The average layer thickness of the coating was approximately 15 to 20 μm.

The following solutions were used for coating the stents:
0.75% P(HB-HV) with 25% HV in DMAA or THF;
0.75% P(HB-HV) with 25% HV+0.5% by weight shikonin (based on the weight of P(HB-HV)) in DMAA or THF;

The stents were coated by several times being immersed into the various solutions.

In addition, polymer films from the indicated solutions (P(HB-HV) with or without shikonin) were tested on stainless steel plates from the same solutions with a layer thickness of 20 to 30 μm. The surface of an uncoated stent served as control.

Preparation of the Blood

Whole blood from healthy adult donors was put into siliconized glass vessels. Blood clotting of 10 ml of blood was prevented by adding sodium citrate in a ratio of (blood: sodium citrate) 9:1. Blood platelet-enriched plasma was obtained by centrifuging the whole blood at 100×g for 20 minutes at room temperature. The blood platelet-enriched plasma fraction was removed with a plastic pipette and immediately employed in the experiments.

50 μl drops of this blood platelet-enriched plasma fraction were put on the surfaces of the plate samples and incubated for 15 min. To test the coated stents, they were put in a vessel with blood platelet-enriched plasma and incubated for 30 minutes. The number of blood platelets which adhere to the surface during this period was sufficient for a qualitative analysis, because the platelets formed no large thrombus-like structures. The samples were washed with physiological saline solution in order to wash off unadsorbed plasma proteins and weakly adhering blood platelets. The samples were then fixed in 2.5% glutaraldehyde, and subsequently dehydrated by standard techniques with an increasing ethanol content.

Adhesion of the blood platelets was investigated by scanning electron microscopy (SEM) (JSM T330, JEOL, Japan). All the samples were made conductive by coating of copper with 1.2 kV, 10 mA for 7 minutes (JEOL JFC-1100, Japan). The microscopic investigations were carried out with a voltage of 5 kV. 25 sections each 400 μm² in size were selected at random for each sample. The qualitative total blood platelet number Nₜₒₜ and the number of blood platelets N_{I} of the following two categories of cells which explicitly reflect the activation of the blood platelets were then evaluated. Each category comprises two morphological classes of adherent blood platelets:
Ia) single, non-activated or only slightly activated deformed cells;
Ib) pseudopod-forming cells or cells in an early stage of spreading.
IIa) fully spread blood platelets;
IIb) aggregates (of two or more blood platelets).

With reference to this classification, blood platelets from category I interact only weakly with the surface; in contrast thereto, a strong interaction takes place between the surface and the blood platelets of category II.

Results

A) Coating with a 0.75% by Weight Solution of P(HB-HV) and P(HB-HV)+Shikonin in DMAA

The number of cells on the uncoated stents proved to be very low, and where adherent blood platelets were present virtually all the cells were completely spread on the uncoated stent. The total number on the P(HB-HV) film was higher than that for the control and all morphological classes of the cells were present on this surface, but the number of aggregates proved to be very low.

Shikonin by contrast was able distinctly to reduce the amount of adherent blood platelets compared with films coated only with P(HB-HV). In this case, only two morphological classes of cells of category I were observable: slightly activated blood platelets and pseudopods-forming cells.

It is evident on the basis of these results that P(HB-HV)-Sh films are more suitable than surfaces with pure P(HB-HV) because the shikonin-containing surfaces showed a lower affinity for cell binding.

In order to determine the connection between blood platelet activation and shikonin concentration, the blood platelet adhesion to films of pure P(HB-HV), of P(HB-HV) with 0.1% by weight shikonin (P(HB-HV)-0.1 Sh) and of P(HB-HV) with 0.5% by weight shikonin (P(HB-HV)-0.5 Sh) was determined for an incubation time of 15 minutes and 30 minutes. It was possible to demonstrate thereby that addition of shikonin was able to reduce the number and the degree of activation of the blood platelets.

b) Coating with a 0.75% by Weight Solution of P(HB-HV) and P(HB-HV)+Shikonin in THF

The coatings were applied as multilayer films to expanded and unexpanded stents. The stents were coated with the polymer by immersing it in the diluted working polymer solution (about 2 to 4 times). Each layer was then dried with hot air.

A comparative analysis of the adhesion of blood platelets to the uncoated stent, to a stent coated with diamond-like carbon and to a stent coated with P(HB-HV)-0.5 Sh was carried out. The incubation time of the samples in plasma enriched with blood platelets was from 15 to about 30 minutes.

No blood platelets were observed on the stents coated with P(HB-HV)-0.5 Sh. However, in the same time it was possible to find blood platelets on the uncoated and on the carbon-coated stents. This means that the properties of shikonin in relation to the activation of blood platelets are also retained on use of THF as solvent.

3. Determination of the Biological Properties of the Films Produced

These investigations were carried out in accordance with the international standard for biological investigations on medical devices ISO 10993 and in accordance with the national standard GOST R ISO 10993.

a) Determination of the Hemolytic Properties

The hemolysis was determined by preparing extracts of the materials, in particular of P(HB-HV) and of P(HB-HV)-0.5 Sh, in each case in physiological saline solution. Whole blood was added to these extracts, followed by incubation at 37° C. for one hour.

The extracts were removed, the mixture was centrifuged (50 minutes at 400×g) and the cell-free supernatant was carefully removed. The hemoglobin concentration for this supernatant was determined by photometry, and the hemolytic index (%) was calculated from the ratio of liberated hemoglobin to hemoglobin present. A pure saline solution was used as control in this case.

Under the given conditions, both extracts, that is to say the extract of P(HB-HV) and that of P(HB-HV)-Sh, proved to be nonhemolytic (hemolytic index: 0%).

b) Determination of the Complement System Activation

The compatibility of the polymer films with blood was tested by determining the hemolytic activity of the complement system of human blood plasma before and after its incubation with a film sample or extract. The tested samples were films of P(HB-HV), P(HB-HV)-Sh and of cuprophane (control).

The concentration of the hemoglobin liberated by the lytic effect of the complement system from sheep erythrocytes coated with antibodies reflects the complement activity in the serum sample. The parameter for the assay was the time required for 100 μl of the tested serum to lyze (π_{1/2}) 50% of 5 ml of sensitized sheep erythrocytes (5×10⁸ cells/ml). The temperature of the reaction mixture was kept constant at 37° C. by means of thermostats.

Human serum was first diluted with a buffer (comprising 0.15 mM CaCl₂ and 0.5 mM MgCl₂) and then incubated with or without polymer samples at 37° C. for 60 min. The values for the remaining complement activity ((CH₅₀)^{R}%) were determined as criterion of the activating properties of the polymers in the following way:
(CH₅₀)^{R}ᵢ=(π_{1/2})₀/(π_{1/2})(t)ᵢ(c) 100%
where:
(π_{1/2})₀=time for 50% lysis of sheep erythrocytes by the serum complement before incubation with the sample;
(π_{1/2})(t)ᵢ=time for 50% lysis of sheep erythrocytes by the serum complement after incubation time t with the polymer sample;
(π_{1/2})(t)_{(c)}=time for 50% lysis of sheep erythrocytes by the serum complement after incubation time t in the control (without polymer sample).

The results of these tests are summarized in table II below:

| Tested samples | CH₅₀ (n = 3) |
|---|---|
| Cuprophane (control material) | 89 ± 5 |
| P(HB-HV) extract | 85 ± 5/80 ± 5 |
| P(HB-HV)-Sh extract | 90 ± 5/85 ± 5 |
| Serum after 1 h incubation | 98 ± 5 |
| (control serum) | |

As is evident from the table, the complement activity of serum incubated with P(HB-HV)-Sh extract proved to be lower than that of serum incubated with pure P(HB-HV) extract. In addition, it showed approximately the same CH₅₀ as cuprophane.

C) Intramuscular Implantation Test

Films of P(HB-HV) only and of P(HB-HV)-Sh were implanted in accordance with ISO 10993 Part 6 into the muscles of guinea pigs. The size of the film samples was 0.5×1 cm with a thickness of 20 to 30 μm. For each type of sample, two guinea pigs with the implant were observed for 7 days.

It was shown after 7 days that fibroblast monolayers formed around the P(HB-HV) and P(HB-HV)-Sh films. Infiltration of macrophages and lymphocytes around these implants proved to be a typical tissue response after implantation for 7 days. It was nevertheless possible to show that the degree of the inflammatory response to the biodegradable polymer with shikonin was less than with the pure P(HB-HV) film.

### SUMMARY

With the results it could be shown that firstly the composition of 25% by weight polyhydroxyvalerate and 75% by weight polyhydroxybutyrate proved to be an optimal composition for the coating, and that this mixture together with a particular shikonin content showed high adhesion to metallic surfaces. In addition, it could be shown that the properties of the polymer coating were not affected by repeated expansion of the coated stent.

Further, it cold be shown that a concentration of shikonin of up to 0.5% by weight inhibited virtually completely the adhesion of human blood platelets to the polymer surface in vitro. Thus, shikonin can be used in suitable concentrations for preventing excessive cell proliferation.

A comparative study on the biological properties of a film of pure P(HB-HV) or of P(HB-HV)-Sh in THF in vitro showed that shikonin had good compatible properties in relation to complement activation and blood platelet adhesion.

With the results it could further be shown that a smooth, thin, fibrocellular layer is formed around the stent through a coating with P(HB-HV)-Sh, leading to reendothelialization with an adequate diameter for normal blood flow.

1. A coating composition for an implantable medical device, the coating composition comprising at least one polymer and at least one bioactive agent, characterized in that the bioactive agent is naphthoquinone and/or a naphthoquinone derivative.

2. The coating composition as claimed in claim 1, characterized in that the naphthoquinone derivative is naphthazarin and/or a naphthazarin derivative.

3. The coating composition as claimed in claim 1 or 2, characterized in that the derivative is selected from the group comprising shikonin, alkannin, arnebin.

4. The coating composition as claimed in any of claims 1 to 3, characterized in that the derivative is shikonin.

5. The coating composition as claimed in any of claims 1 to 4, characterized in that naphthazarin and/or naphthazarin derivatives are present in the content of from 0.01 to 1% by weight, preferably of 0.5% by weight.

6. The coating composition as claimed in any of claims 1 to 5, characterized in that the polymer is an absorbable polyester.

7. The coating composition as claimed in claim 6, characterized in that the absorbable polyester is selected from the group comprising polyglycolic acid, polylactic acid, polycaprolactone, polyhydroxyalkanoate and copolyesters thereof.

8. The coating composition as claimed in claim 7, characterized in that the polyhydroxyalkanoate is selected from the group comprising polyhydroxybutyrate, polyhydroxyvalerate and copolyesters thereof.

9. The coating composition as claimed in claim 8, characterized in that the copolyester is a polyhydroxybutyrate-polyhydroxyvalerate copolyester.

10. The coating composition as claimed in claim 9, characterized in that in the copolyester polyhydroxyvalerate is present in a content of from 20 to 30% by weight, preferably of 25% by weight, and polyhydroxybutyrate is present in a content of from 70 to 80% by weight, preferably of 75% by weight.

11. The coating composition as claimed in any of claims 1 to 10, characterized in that the absorbable polymer and naphthoquinone and/or naphthoquinone derivative are present dissolved in at least one solvent.

12. The coating composition as claimed in claim 11, characterized in that the solvent is dimethylacetamide and/or tetrahydrofuran.

13. A method for coating an implantable medical device comprising the steps:
(a) applying the coating composition as claimed in any of claims 1 to 12 onto the implantable medical device,
(b) drying of the implantable medical device and
(c)—where necessary—repeating steps (a) and (b).

14. The method as claimed in claim 13, characterized in that the coating composition is sprayed on.

15. The method as claimed in claim 13, characterized in that the coating composition is applied by immersing the implantable medical device into the coating composition.

16. The method for coating an implantable medical device as claimed in any of claims 13 to 15, characterized in that a coating composition which includes a copolyester of polyhydroxybutyrate-polyhydroxyvalerate in a ratio of 3:1 and shikonin in a content of from 0.01 to 1% by weight, in particular of 0.4 to 0.6% by weight, is applied.

17. The method for coating an implantable medical device as claimed in any of claims 13 to 16, characterized in that a stent is employed as implantable device.

18. Use of a coating composition as claimed in any of claims 1 to 12 for coating implantable medical devices.

19. Use of naphthoquinone and/or of a naphthoquinone derivative, in particular shikonin, for producing a coating composition for an implantable medical device.

20. Use of naphthoquinone and/or of a naphthoquinone derivative, in particular shikonin, for coating an implantable medical device.

21. An implantable medical device coated with a coating composition as claimed in any of claims 1 to 12.

22. A stent coated with a coating composition as claimed in any of claims 1 to 12.
